(19) United States
(12) Patent Application Publication
(10) Pub. No.: US 2013/0053877 A1
(43) Pub. Date: Feb. 28, 2013

(54) Multiple Function Surgical Instrument

(71) Applicant: IMDS Corporation, Logan, UT (US)

(72) Inventors: Moutaa BenMaamer, Ft. Myers, FL (US); Brad Anthony Niese, Chandler, AZ (US); Dylan Matthew Hushka, Gilbert, AZ (US)

(73) Assignee: IMDS CORPORATION, Logan, UT (US)

(21) Appl. No.: 13/652,645

(22) Filed: Oct. 16, 2012

Related U.S. Application Data
(63) Continuation-in-part of application No. 13/020,239, filed on Feb. 3, 2011.
(60) Provisional application No. 61/301,823, filed on Feb. 5, 2010.
(60) Provisional application No. 61/548,461, filed on Oct. 18, 2011.

(51) Int. Cl. A61B 17/3201

(52) U.S. Cl. 606/174

(57) ABSTRACT

A surgical instrument includes first and second jaw members which are pivotable between closed, grasping, and cutting configurations. A spring-driven safety feature prevents unintentional movement of the first and second jaw members into the cutting configuration. The instrument allows the user to perform separate tissue grasping and cutting actions without changing instruments or removing the instrument from the surgical site.

### CROSS-REFERENCE TO RELATED APPLICATIONS

This application is a continuation-in-part of:

pending U.S. patent application Ser. No. 13/020,239, filed Feb. 3, 2011, which is entitled MULTIPLE FUNCTION SURGICAL INSTRUMENT, which is a non-provisional of:

U.S. Provisional Patent Application No. 61/301,823, filed Feb. 5, 2010, which is entitled MULTIPLE FUNCTION SURGICAL INSTRUMENT AND METHOD.

This application is also a non-provisional of:

pending U.S. Provisional Patent Application No. 61/548,461, filed Oct. 18, 2011, which carries Applicant's docket No. MLI-100 PROV, and is entitled LAPAROSCOPIC DISSECTOR WITH SAFETY FEATURES.

The above-identified documents are incorporated herein by reference.

### FIELD OF THE INVENTION

The present invention relates to an implement for performing minimally invasive surgery and, more particularly, to a multiple function implement for performing a laparoscopic surgery.

### BACKGROUND OF THE INVENTION

Laparoscopic surgery, or laparoscopy, is a minimally invasive surgery inside the abdominal cavity, chest cavity, and/or the joints of a patient, for example. During a typical laparoscopy, the desired cavity of the patient is insufflated, if necessary, with gas, and cannula sleeves, or trocars, are passed through small incisions (approximately one-half inch) to provide entry ports for receiving laparoscopic surgical instruments.

The laparoscopic instruments include a laparoscope (for viewing the surgical field) and other separate instruments for performing a number of different tasks. These instruments are similar to those used in a conventional surgery, except the working end, or effector, of each instrument is spaced from its respective handle by an extension tube. The effector of each instrument may include clamps, graspers or dissectors, scissors, suction or irrigation tubes, and needle holders, for example. The surgeon guides a desired instrument, such as one of the above instruments, through a cannula sleeve to an internal surgical site. The desired instrument is manipulated from outside of the abdominal cavity. The surgeon observes the procedure by means of a monitor that displays an image of the surgical site taken from the laparoscope.

Minimally invasive surgical systems are being developed to overcome some of the disadvantages of open surgeries. Minimally invasive surgical instruments deny the surgeon the flexibility of implement placement found in open surgery. Most current laparoscopic implements have rigid shafts. The rigid shafts may create difficulty in approaching the planned surgical site through a small incision. The length of many instruments reduces the ability of a surgeon to feel forces exerted by tissues and organs on the end effector of the implement that is in use, and may results in lack of dexterity and sensitivity. Time and concentration may be lost during the change from one instrument to another. Also, most instruments have only one function, and this is a major impediment to the expansion of minimally invasive surgery.

It would be desirable to produce an instrument for use during minimally invasive surgery that will minimize or eliminate the time required between to change surgical instruments.

### SUMMARY OF THE INVENTION

Concordant and congruous with the present invention, instruments for use during minimally invasive surgery that will minimize the time required between the change of surgical functions of the instrument are disclosed.

In an embodiment of the invention, an instrument used in laparoscopic surgery comprises a trocar having a hollow interior and a sharpened end; and at least two implements having working distal ends and associated shafts extending from the distal ends and terminating in respective handles at proximal ends thereof disposed within the hollow interior of the trocar, wherein the implements may be selectively advanced and retracted by an appropriate force applied to the respective handles of the implements.

In another embodiment of the invention, an instrument used in laparoscopic surgery comprises a trocar having a hollow interior and a sharpened end; and at least three implements having working distal ends and associated shafts extending from the distal ends and terminating in respective handles at proximal ends thereof, wherein the implements are frictionally engaged within the hollow interior of the trocar and may be selectively advanced and retracted by an appropriate force applied to the respective handles of the implements.

In another embodiment of the invention, a surgical instrument for grasping and cutting of tissue comprises a first jaw having a first blade portion; a second jaw having a second blade portion; and a handle portion connected to the first and second jaws, the handle portion actuable to move the first and second jaws between a closed configuration and an open configuration, wherein when the first and second jaws are in the open configuration, a gap is formed between the first and second blade portions to permit receiving of tissue between the first and second blade portions for cutting of tissue; wherein the handle portion comprises a safety feature which prevents unintentional movement of the first and second jaws into the open configuration, the safety feature comprising a spring, the spring captured in a recess formed in the handle portion.

In another embodiment of the invention, a surgical instrument for grasping and cutting of tissue, comprises a first jaw having a first grasping portion and a first blade portion; a second jaw having a second grasping portion and a second blade portion; a handle portion connected to the first and second jaws, the handle portion actuable to move the first and second jaws between a grasping configuration and an open configuration, wherein when the first and second jaws are in the open configuration, a cutting gap is formed between the first and second blade portions to permit receiving of tissue between the first and second blade portions for cutting of tissue; wherein the handle portion comprises a safety feature which prevents unintentional movement of the first and second jaws into the open configuration, the safety feature comprising a spring.

### BRIEF DESCRIPTION OF THE DRAWINGS

The above, as well as other objects and advantages of the invention, will become readily manifest to those skilled in the art from reading the following detailed description when considered in light of the attached drawings. It is appreciated that these drawings depict only typical embodiments of the invention and are therefore not to be considered limiting of its scope.

FIG. 1 is an elevational view, partly in section, of a multifunctional instrument for use in minimally invasive surgery embodying the features of the present invention;

FIG. 2 is a perspective view of a surgical instrument for grasping and cutting of tissue, the instrument including a handle portion, a shaft portion, and a working end, the working end including first and second jaw members;

FIG. 3 is an exploded view of the instrument of FIG. 2;

FIG. 4 is an enlarged side view of the handle portion and a section of the shaft portion of the instrument of FIG. 2, with a handle housing removed to allow visibility of the remainder of the components of the instrument, the instrument in a closed configuration;

FIG. 5 is an enlarged view of the working end and a section of the shaft portion of the instrument of FIG. 2, the instrument in the closed configuration;

FIG. 6 is a side perspective view of first and second jaw members of the working end of FIG. 2;

FIG. 7 is an opposite side perspective view of the jaw members of FIG. 6;

FIG. 8 is a top view of the jaw members of FIG. 6;

FIG. 9 is a partially exploded side view of a section of a drive rod of the shaft portion and the first and second jaw members of the instrument of FIG. 2,

FIG. 10 is a side view of the drive rod and first and second jaw members of FIG. 9, the first and second jaw members translatably connected to the drive rod;

FIG. 11 is a side view of the drive rod and first and second jaw members of FIG. 10, a spacer encircling the drive rod and pivotably connected to the first and second jaw members;

FIG. 12 is a side view of the drive rod, first and second jaw members and space of FIG. 11, an outer shaft encircling the drive rod and the spacer and pivotably connected to the first and second jaw members;

FIG. 13 is an enlarged side view of the handle portion and a section of the shaft portion of the instrument of FIG. 2, with a handle housing removed to allow visibility of the remainder of the components of the instrument, the instrument in a grasping configuration;

FIG. 14 is an enlarged view of the working end and a section of the shaft portion of the instrument of FIG. 2, the instrument in the grasping configuration;

FIG. 15 is an enlarged side view of the handle portion and a section of the shaft portion of the instrument of FIG. 2, with a handle housing removed to allow visibility of the remainder of the components of the instrument, the instrument in a cutting configuration; and

FIG. 16 is an enlarged view of the working end and a section of the shaft portion of the instrument of FIG. 2, the instrument in the cutting configuration.

### DETAILED DESCRIPTION OF THE PREFERRED EMBODIMENTS

The following detailed description and appended drawings describe and illustrate various exemplary embodiments of the invention. The description and drawings serve to enable one skilled in the art to make and use the invention, and are not intended to limit the scope of the invention in any manner. In respect of the methods disclosed, the steps presented are exemplary in nature, and thus, the order of the steps is not necessary or critical.

In this specification, standard medical directional terms are employed with their ordinary and customary meanings. Superior means toward the head. Inferior means away from the head. Anterior means toward the front. Posterior means toward the back. Medial means toward the midline, or plane of bilateral symmetry, of the body. Lateral means away from the midline of the body. Proximal means toward the trunk of the body. Distal means away from the trunk.

In this specification, a standard system of three mutually perpendicular reference planes is employed. A sagittal plane divides a body into bilaterally symmetric right and left portions. A coronal plane divides a body into anterior and posterior portions. A transverse plane divides a body into superior and inferior portions.

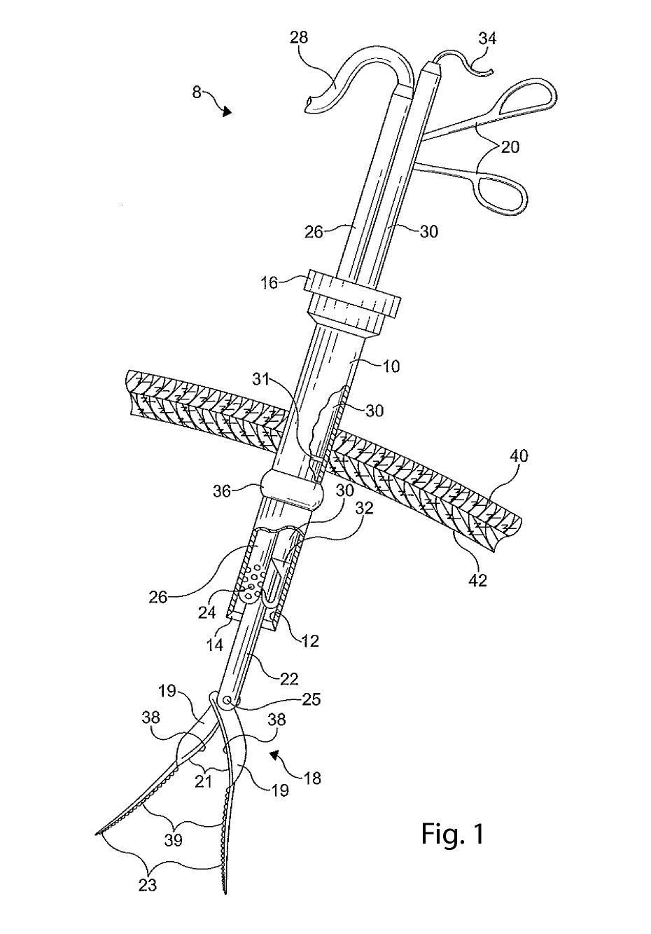

Referring to FIG. 1, there is shown a multiple-function surgical instrument 8 including a trocar 10 having a hollow interior 12 and a sharpened distal end edge 14. The proximal end 16 of the trocar 10 is designed to typically receive a number of individual implements for use in surgical procedures. The proximal end 16 includes a gasket or a seal to provide a seal between the trocar 10 and the individual implements. In FIG. 1, the implements include a dissector/scissors implement 18 equipped with an operating looped handle 20 at the proximal end of a shaft 22. The dissector/scissors implement 18 is disposed at the distal or working end of the shaft 22.

The dissector/scissors implement 18 includes a pair of opposing pivotally-connected members 19. Each of the pair of members 19 includes a first portion 21 for cutting and a second portion 23 for grasping. The first portions 21 of the members 19 are disposed closest to a pivotal point 25 of the dissector/scissors implement 18. Each of the first portions 21 includes blades 38. Each of the second portions 23 includes ridges or serrations 39.

Another implement within the trocar 10 is a perforated irrigator/suction implement 24 at the distal end of a hollow shaft 26. The proximal end of the shaft 26 is coupled to a source of irrigation or suction (not shown) as determined by the surgeon, typically through a tube 28.

A third implement is included in the illustrated embodiment that consists of a shaft 30 having a hook implement 32 at a distal end thereof and an electrical lead 34 at a proximal end thereof. It will be understood that the hook implement 32 is electrically coupled to the electrical lead 34, which, in turn, is connected to a source of electrical energy (not shown).

To prevent undesired contact between the implements 18, 24, 32, each implement 18, 24, 32, and each associated shaft 22, 26, 30, respectively, may be housed in a retractable sheath (not shown). The sheaths of each implement 18, 24, 32 may be retracted or advanced by manipulation of each sheath at a proximal end of the multiple-function surgical instrument 8.

It will be understood that in use, the implements 18, 24, 32 are disposed within the trocar 10 in a retracted position, as the irrigator/suction implement 24 and the hook implement 32 are illustrated in FIG. 1. The sharpened end edge 14 of the trocar 10 may then be inserted into the abdominal cavity of a patient, typically through a small incision in the abdominal cavity, causing the end edge 14 of the trocar 10 to enter the abdominal cavity of the patient through the abdominal wall, comprised of skin 40 and the adjacent muscle 42.

The implements 18, 24, 32 are frictionally engaged within the hollow interior of the trocar 10. The implements 18, 24, 32 may be selectively advanced and retracted by an appropriate force applied by the surgeon to the proximal end of the desired implement 18, 24, 32. In the event it is deemed necessary or desirable to vary the frictional engagement of the implements 18, 24, 32 within the trocar 10, a flexible member 31 may be inserted to extend around the shafts of the implements 18, 24, 32. The flexible member 31 may be a gasket or ring disposed around or formed on the shafts of the implements 18, 24, 32, or the flexible member 31 may be disposed around or formed on each shaft individually. It is understood that the flexible member 31 may be disposed around any combination of the implements 18, 24, 32, as desired. The flexible member 31 may be any member adapted to elastically deform when disposed on the trocar 10, such as an annular spring, for example.

As shown in FIG. 1, a portion of an exterior of the trocar 10 includes a selectively expandable member 36 disposed thereon adjacent the distal end thereof. The expandable member 36 is formed from an elastic material adapted to be inflated or expanded. Once the expandable member 36 of the trocar 10 is inserted into the abdominal cavity of the patient, the expandable member 36 may be elastically expanded to prevent the removal of the trocar 10 from the abdominal cavity during a procedure. A source of fluid (not shown), such as a hand pump, in communication with the expandable member 36 may be used to selectively inflate and deflate the expandable member 36. It is understood that the fluid used to inflate the expandable member 36 may be a gas or a liquid, such as a saline solution, for example, as desired.

It will be appreciated that the afore-described instrument 8 may incorporate a number of different implements readily available for selective use by the surgeon without requiring a request to an accompanying nurse or other medical provider for another implement, thereby allowing the surgeon's concentration to be continuous without a break, resulting in increased efficiency and reduced operating time. Additional implements incorporated into the multiple-function surgical instrument 8 may include clamps, needle holders, a camera, and a light, for example. The consequence results in manifest benefits to the patient.

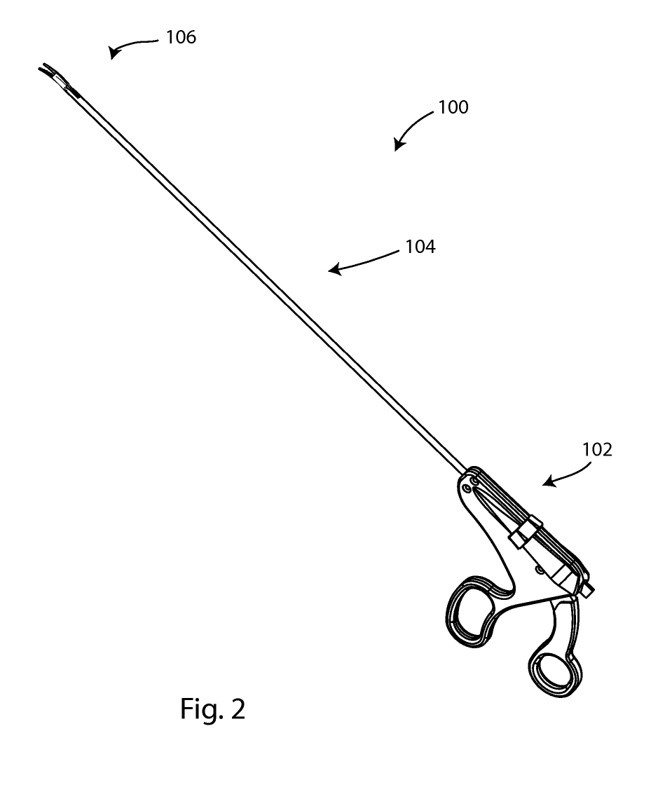

Another embodiment of a dissector/scissors implement is shown in FIGS. 2-16. Instrument 100 includes a set of pivotable jaws which include both cutting and grasping portions. In a first mode or configuration, the instrument functions as a grasper. The instrument includes a spring-driven force override to permit transition into a second mode wherein the instrument functions as a tissue cutter. The spring serves as a force detent that the surgeon must override in order to move the jaws past the grasping mode and into the cutting mode. In another configuration, the instrument is closed with the pivotable jaws shut against one another.

Referring to FIG. 2, dissector 100 includes handle portion 102, shaft portion 104, and working end 106. The handle portion 102 is actuable to move working end 106 between the cutting and grasping modes.

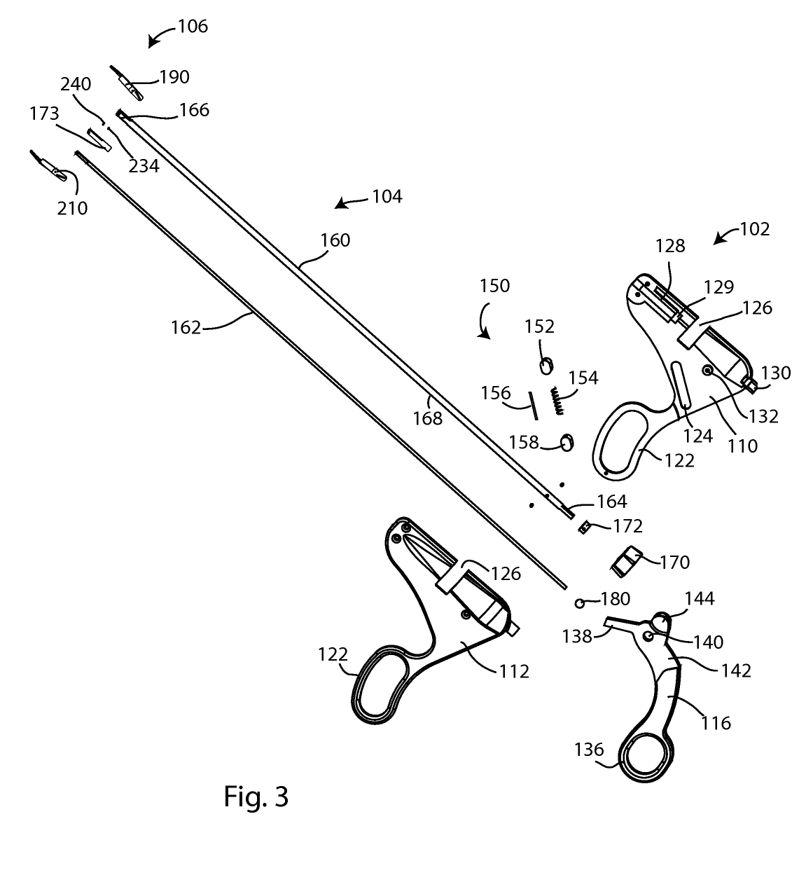

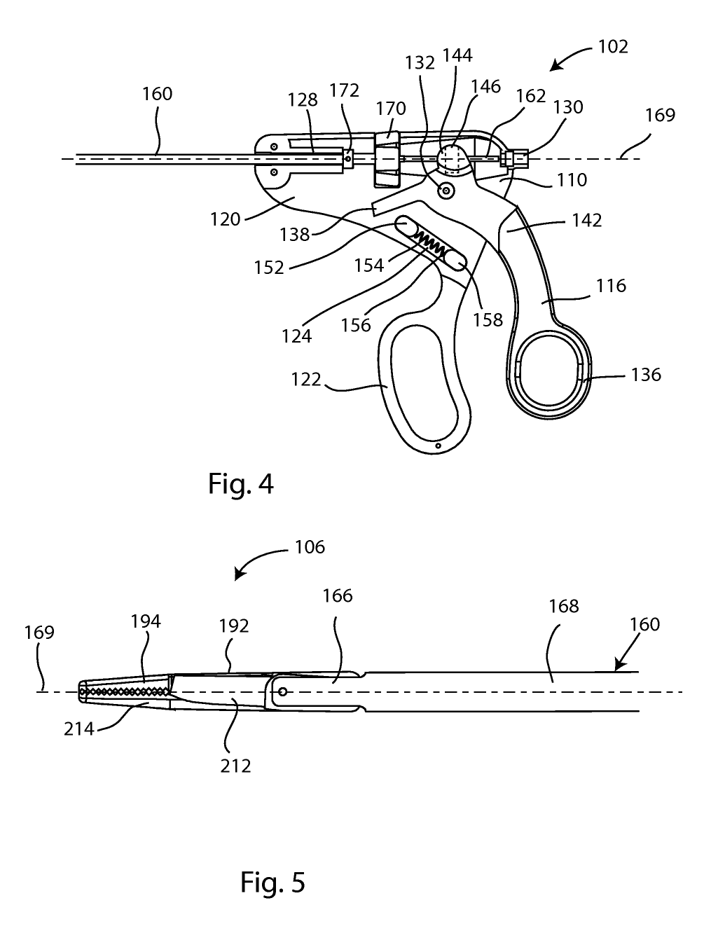

Referring to FIGS. 3 and 4, handle portion 102 includes a first handle housing 110 and a second handle housing 112, which may be mirror images of one another. The housings may be injection molded, and may be snapped, press fitted, screwed or otherwise fastened together to form a handle 114. A driver lever 116 is partially received in the handle 114. First handle housing 110 will be described in detail; it is understood that second handle housing 112 includes the same features in a mirror-image configuration. First handle housing 110 includes a housing wall 120; a finger loop 122 projects generally inferiorly from the housing wall. An elongated recess 124 is formed as a recess in the housing wall 120, and is shaped as an elongated oval with a straight portion between the ends of the oval. The straight portion forms a track to allow smooth translation of a slider within the recess. A notch 126 is formed in an edge of the handle housing 110. A shaft retention feature 128 is formed on the housing wall 120, adjacent a collar recess 129 in the housing wall 120. An opening 130 is formed at the proximal end of the handle housing 110, and may function as an access port for an electrical lead and/or a tube for suction or irrigation. A boss 132 projects interiorly from the housing wall 120 to provide a pivot point for the driver lever 116.

The driver lever 116 includes an actuator formed as a finger loop 136 which projects proximally from the handle 114 when the instrument 100 is properly assembled. A lever tip 138 projects distally from the lever 116. Intermediate the finger loop 136 and the lever tip 138, a pivot hole 140 is formed to receive bosses 132. A lever shaft 142 extends between and connects the finger loop 136 to the remainder of the lever. The perimeter of the handle 114, the lever shaft 142 and the finger loops 122, 136 may be curved or otherwise shaped to form an ergonomic gripping configuration for a user's hand. A forked shaft connection feature 144 is formed on the driver lever 116, and includes a retention slot 146 positioned between the forks of the connection feature. In other embodiments, the relative positions of the features of the lever may vary; for example the finger loop may project distally from the handle. In other embodiments, the actuator may take another form such as a trigger, button, tab, or slide.

Referring to FIGS. 3, 4, 13, and 15, a safety feature 150 includes a slider 152, spring 154, spring rod 156 and stop 158. As seen in FIG. 4, slider 152 and stop 158 are received in opposite ends of recess 124. Spring rod 156 extends between the slider and the stop, and spring 154 is received around and captured by spring rod 156. Slider 152 includes a longitudinal bore in which spring rod 156 is received. Recess 124 functions as a track in which the slider 152 can translate. During use of the instrument, force may be exerted on driver lever 116 to pivot lever 116 to permit lever tip 138 to contact and urge translation of slider 152 in recess 124 in a first direction, toward stop 158. When the force is release, the spring bias of spring 154 urges the slider to translate in recess 124 in a second direction opposite the first direction, away from stop 158.

The shaft portion 104 of instrument 100 includes a tubular outer shaft 160 and a drive rod 162 which is partially received in outer shaft 160. The outer shaft 160 includes a proximal portion 164, a distal portion 166, and a shaft portion 168 extending between and connecting the proximal and distal portions 164, 166. Both the proximal and distal portions 164, 166 may be forked. A wheel 170 is captured in notch 126. The forked proximal end 164 of the outer shaft 160, and the drive rod 162 extend through an opening in the wheel 170. The outer shaft 160 and drive rod 162 are coaxial, sharing a drive axis 169 defined by the drive rod. A collar 172 encircles outer shaft 160 near the proximal portion 164; the collar 172 is rotatably captured in the collar recess 129 formed in the first housing wall 120. The wheel 170 may be rotated about the drive axis 169 of the outer shaft 160 to rotate the outer shaft 160, rod 162 and the working end 106 a full 360°. Translation of the outer shaft 160 is prevented by the capture of the collar 172 in the collar recess 129: the collar 172 and outer shaft 160 can rotate relative to the collar recess 129 and the handle portion 102, but cannot translate.

Drive rod 162 extends through a ball 180 which is captured in retention slot 146 of driver lever 116. The ball 180 is fixedly attached to the drive rod; the drive rod 162 cannot translate relative to the ball. The ball 180, and attached drive rod 162, can rotate within retention slot 146, but cannot translate relative to the retention slot 146. Actuation of driver lever 116 translates drive rod 162 along drive axis 169.

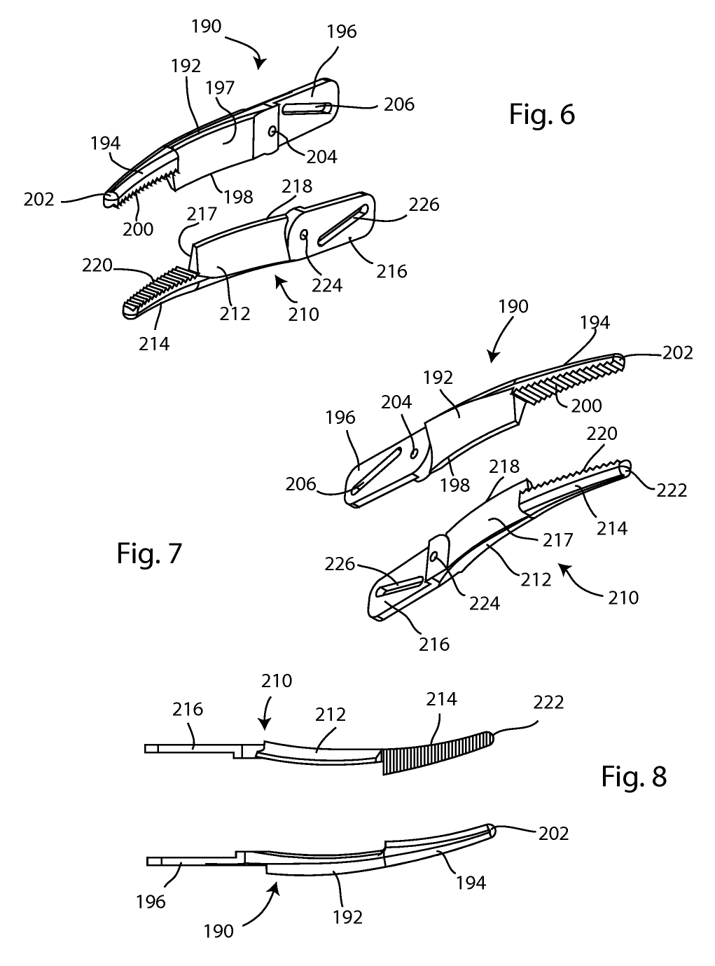

Referring to FIGS. 6-8, working end 106 includes a first jaw 190 and a second jaw 210. In the embodiment shown, first and second jaws 190, 210 are curved; in other embodiments they may be straight, jogged, offset, hollow, or another shape. In the embodiment shown, first and second jaws 190, 210 are identical to one another with the exception of their curvatures relative to one another. First jaw 190 includes a first portion which is a blade portion 192, a second portion which is a grasping portion 194, and a third portion which is a connection portion 196. The grasping portion 194 is distal to the blade portion 192. The blade portion 192 includes a blade surface 197 which terminates at a cutting edge 198 which may be beveled. The blade portion 192 may have a triangular profile when viewed from the proximal or distal perspective or in transverse cross section. The grasping portion 194 includes a grasping surface 200 which may be ridged or serrated to provide optimal grip of tissues, and a distal tip 202 which may be rounded or blunt to permit unimpeded passage into or between tissues. The grasping surface 200 is offset from the cutting edge 198, and the grasping surface may be perpendicular to the blade surface 197. The connection portion includes a pin hole 204 which forms a pivot point for the first jaw 190, and a jaw slot 206 which permits actuation of the jaw via the drive rod. Blade and grasping portions 192, 194 are curved whereas connection portion 196 is straight.

Second jaw 210 includes features which are similar to those of first jaw 190, including: blade portion 212, grasping portion 214, and connection portion 216. Blade portion 212 includes blade surface 217 and cutting edge 218, and grasping portion 214 includes grasping surface 220 and distal tip 222. Connection portion 216 includes pin hole 224 and jaw slot 226. In some embodiments, the jaw grasping portions and/or tips may be upturned, downturned, pointed, divergent, or another configuration.

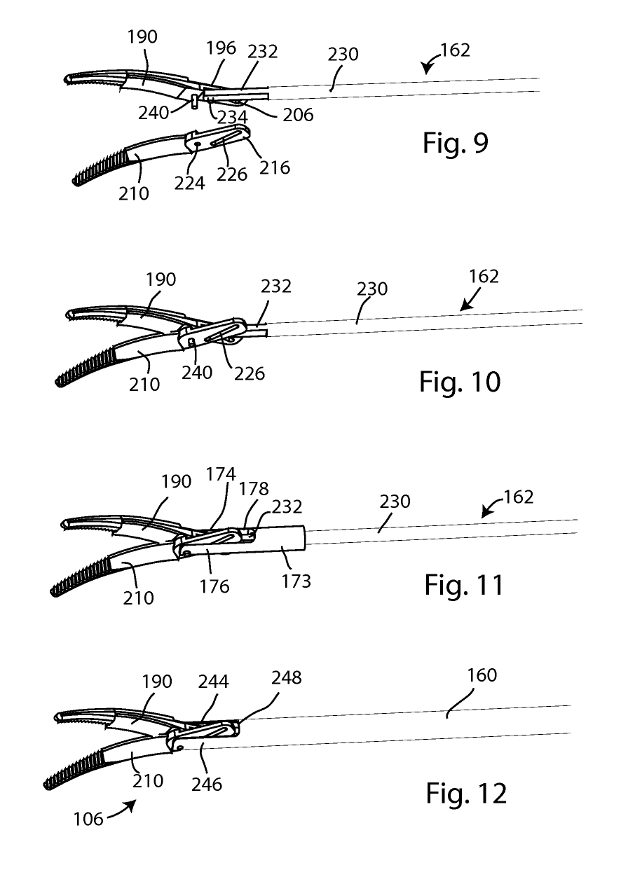

FIGS. 9-12 illustrate the assembly of first and second jaws 190, 210 with drive rod 162, outer shaft 160 and a spacer 173. Other embodiments may not include a spacer. The drive rod 162 includes a shaft portion 230, and a rod extension 232 distal to the shaft portion. In the embodiment shown, the rod extension 232 is reduced in width from the shaft portion 230 to allow the combined widths of the rod extension 232 and blade connection portions 196, 216 to be received in the spacer 173. A drive pin 234 extends through the rod extension 232, protruding on both sides of the rod extension. As seen in FIGS. 9 and 10, first jaw 190 is connected to the drive rod 162 via mounting of jaw slot 206 on drive pin 234. A pivot pin 240 extends through pin hole 204. Second jaw 210 is mounted onto the opposite side of rod extension 232, with drive pin 234 received in jaw slot 226, and pivot pin 240 extending through pin hole 224. With jaws 190, 210 thus mounted on drive rod 162, the jaws 190, 210 can pivot about pivot pin 240. When drive rod 162 is axially translated, the jaws 190, 210 are pivotably scissored together as drive rod 162 moves proximally and translates drive pin 234 proximally toward the proximal ends of slots 206, 226. Jaws 190, 210 scissor apart as drive rod 162 moves distally and translates drive pin 234 distally toward the distal ends of slots 206, 226 as seen in FIG. 10. The axial translation of the driver rod 162 is limited by the jaw slots 206, 226.

Referring to FIG. 11, the spacer 173 encircles the distal end of drive rod 162, and aligns the drive rod 162 and jaws 190, 210 to the distal end of the outer shaft 160. The spacer may also aligns jaws 190, 210 relative to one another for proper cutting action. Spacer 173 includes first and second spacer prongs 174, 176 which define a U-shaped spacer passage 178. First and second spacer prongs 174, 176 of spacer 173 extend on the outer sides of jaw connection portions 196, 216. Pin 240 extends through pin holes in each of the spacer prongs 174, 176, allowing the jaws 190, 210 to pivot within the spacer passage 178. Referring to FIG. 12, outer shaft 160 encircles spacer 173 and drive rod 162, with spacer 173 positioned between drive rod 162 and outer shaft 160. Outer shaft 160 includes first and second shaft prongs 244, 246 which extend parallel to and outside of the spacer prongs 174, 176. Pin 240 extends into pin holes in each of the shaft prongs 244, 246 to fasten the outer shaft 160 to the spacer 173 and the jaws 190, 210. Because the outer shaft 160 is fastened to the working end 106, rotation of the outer shaft 160 by wheel 170 also rotates the working end 106.

FIGS. 4, 5 and 13-16 illustrate the working of instrument 100. FIGS. 4 and 5 show the instrument 100 in the closed mode or configuration. In the closed mode, finger loops 122, 136 are at their closest point together, and lever tip 138 is pivoted away from slider 152. Grasping portions 194, 214 are closed against one another, the grasping surfaces are substantially parallel to one another, and blade portions 192, 212 entirely overlap one another. Drive rod 162 is translated to its proximal extreme.

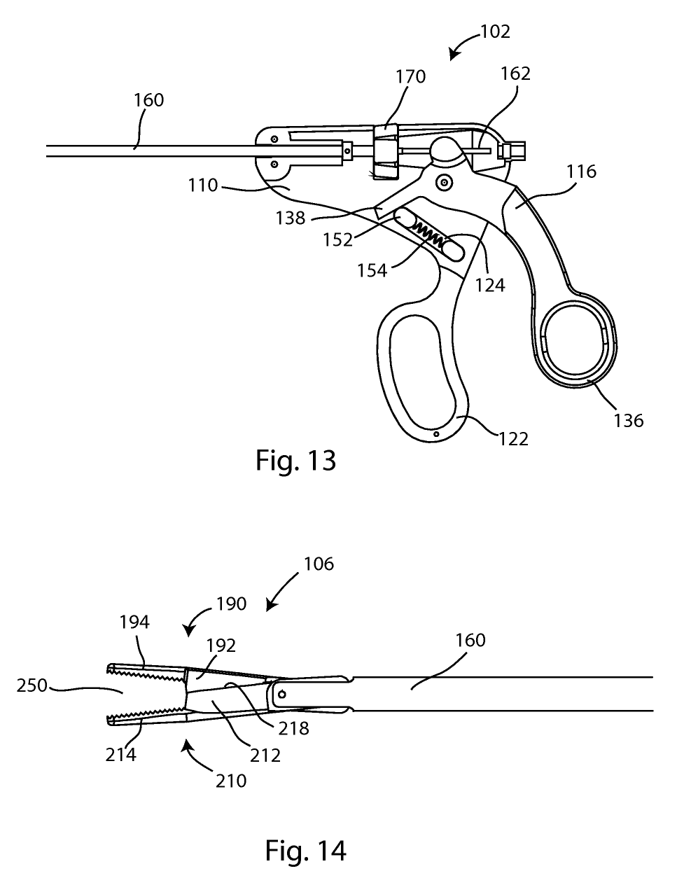

Referring to FIGS. 13 and 14, the instrument 100 is shown in the grasping mode or configuration. In the grasping mode, driver lever 116 is pivoted so that finger loops 122, 136 are farther apart than in the closed mode, and drive rod 162 is translated distally. The lever tip 138 may touch slider 152, but spring 154 is not compressed and slider 152 is at the distal end of recess 124. At the working end 106, the translation of drive rod 162 has pivoted jaws 190, 210 so that the grasping portions 194, 214 are no longer closed together, and tissue may be grasped in a grasping gap 250 formed between them. However, the jaws 190, 210 are not pivoted so far that a gap is formed between the blade edges 198, 218 of blade portions 190, 210. Hence, tissue may be grasped between the grasping portions 194, 214 but not between the blade portions 190, 210. In this mode, slider 152 and spring 154 impede the movement of lever tip 138; the spring bias of spring 154 acts as a safety to prevent unintentional movement into the cutting mode.

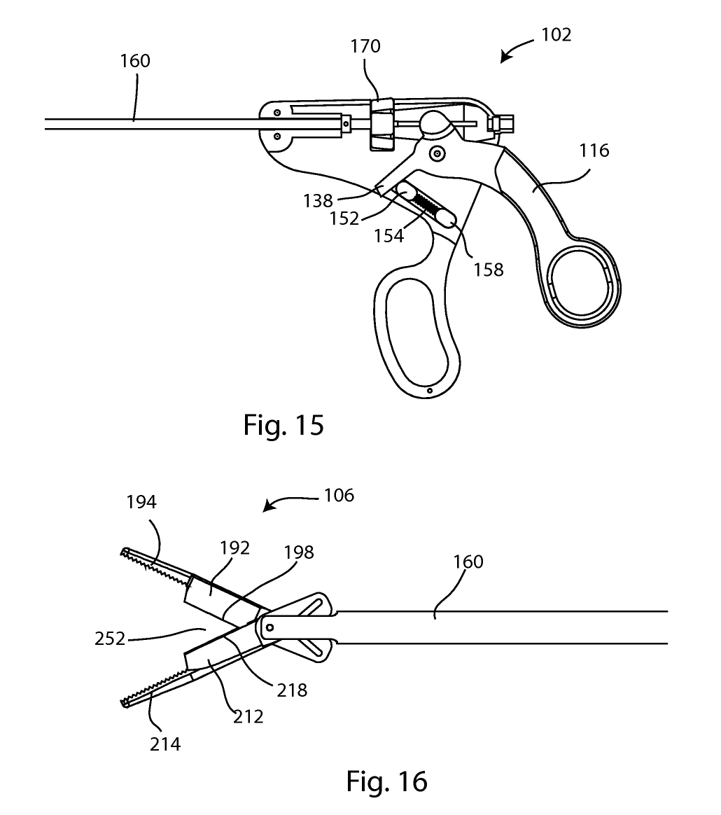

Referring to FIGS. 15 and 16, the instrument 100 is shown in the open, or cutting mode or configuration. In the cutting mode, sufficient force is exerted on driver lever 116 to pivot it so that finger loops 122, 136 are farther apart than in the grasping mode, and drive rod 162 is translated distally. The lever tip 138 is pushing slider 152. The force exerted by lever tip 138 against slider 152 has overcome the spring bias of spring 154, so that spring 154 is compressed toward stop 158. At the working end 106, drive rod 162 is translated distally and jaws 190, 210 are pivoted fully open, so that a cutting gap 252 is formed between blade edges 198, 218. In this mode, tissue can be received in the cutting gap 252 between the blade edges. If force on the driver lever 116 and lever tip 138 is released, the spring bias of spring 154 will push lever tip 138 back toward the grasping or closed modes, translating driver rod 162 proximally and scissoring the blade edges 198, 218 together and cutting the tissue in the cutting gap 252. It is understood that the instrument 100 may be actuated along a continuum between the fully closed mode seen in FIG. 5 and the fully open mode seen in FIG. 14 by varying the force exerted on the driver lever 116.

In one method of use, instrument 100 is used in a laparoscopic procedure to grasp and cut tissues. An incision may be made in the patient's skin and a passage to the targeted tissue is created, alternatively the instrument may be inserted in a natural orifice. In one example, a cannula or other port is inserted to create the passage. The working end 106 of instrument 100 is inserted into the passage until it approaches the targeted tissue. During insertion of the instrument 100, the instrument may be in the closed mode in order to smoothly traverse through the passage; alternatively it may be in the grasping mode. When the working end 106 approaches the targeted tissue, the practitioner pivots driver lever 116 to transform the instrument between the grasping and closed modes to grasp tissue in the grasping gap 250 between the jaw grasping portions 194, 214. For example, the instrument is actuated to the grasping mode, tissue is received in grasping gap 250, and the instrument is actuated toward the closed mode to close the grasping surfaces 200, 220 down on the tissue. The instrument 100 may be moved while tissues are grasped between the jaws, for example to pull tissues aside. The grasped tissue is released by pivoting the driver lever 116 toward the grasping mode to release the tissue from between the grasping surfaces 200, 220. These steps may be repeated to grasp and move other tissues. At any point, wheel 170 may be rotated to rotate the jaws about the drive axis 169. When cutting of tissues is desired, the practitioner exerts additional force on the driver lever 116 to overcome the spring bias and transform the instrument into the cutting mode. Tissue is positioned in the cutting gap 252, and the force on the driver lever 116 is reduced, allowing the spring bias of the spring 124 to move the slider 152 and lever tip 138, and consequently translate the drive rod 162 proximally to close the jaw blades 198, 218 toward one another, severing the tissue in the cutting gap 252 between the jaw blades. The instrument 100 may be actuated between the grasping, closed and cutting modes as desired without withdrawing the instrument from the passage. When grasping and cutting procedures at the targeted site are complete, the instrument may be withdrawn; subsequently the passage may be closed by removal of the cannula or port.

Significantly, the instrument 100 may be deployed in any of the closed, grasping and/or cutting modes, and actuated between the modes, without removal of the instrument from the passage and without repositioning the instrument within the passage. This may result in ease of use for the practitioner; fewer instruments to procure, inventory, and prepare; and a shorter procedure for the patient with less time under anaesthesia.

In some embodiments a monopole cauterization plug may be included and connected to the drive rod through the opening 130 at the proximal end of the handle 114. High frequency electricity may be sent from the monopole plug into the drive rod and electrically energize the first and second jaws 190, 210 to create heat for wound and vessel cauterization. The flow of electricity may be controlled through a separate module that the surgeon controls (usually via foot switch). In this embodiment, the outer shaft 160 is electrically isolated from this energy so as to prevent inadvertent electrical discharge through the shaft of the device. In another embodiment, the electrical lead 34 may be included to perform the same function as the monopole cauterization plug. In another embodiment, the tube 28 may connect to the instrument 100 through opening 130 to provide irrigation or suction through the instrument.

In some embodiments, the housings 110, 112, driver lever 116, wheel 170, slider 152 and stop 158 are made of plastic, and may be injection molded. In other embodiments, the housing and driver lever may be made of stainless steel and may be sterilized and reused. The jaws 190, 210, outer shaft 160 and drive shaft 162 may be made of stainless steel. The drive shaft 162 and jaws 190, 210 may be made of an electrically conductive material, and may be insulated from the outer shaft 160, handle 114 and driver lever 116.

It should be understood that the present system, kits, apparatuses, and methods are not intended to be limited to the particular forms disclosed. Rather, they are to cover all modifications, equivalents, and alternatives falling within the scope of the claims.

The claims are not to be interpreted as including means-plus- or step-plus-function limitations, unless such a limitation is explicitly recited in a given claim using the phrase(s) “means for” or “step for,” respectively.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

The present invention may be embodied in other specific forms without departing from its spirit or essential characteristics. It is appreciated that various features of the above-described examples can be mixed and matched to form a variety of other alternatives. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A surgical instrument for grasping and cutting of tissue, comprising:
a first jaw having a first blade portion;
a second jaw having a second blade portion; and
a handle portion connected to the first and second jaws, the handle portion actuable to move the first and second jaws between a closed configuration and an open configuration, wherein when the first and second jaws are in the open configuration, a cutting gap is formed between the first and second blade portions to permit receiving of tissue between the first and second blade portions for cutting of tissue;
wherein the handle portion comprises a safety feature which prevents unintentional movement of the first and second jaws into the open configuration, the safety feature comprising a spring, the spring captured in a recess formed in the handle portion.

2. The surgical instrument of claim 1, wherein the handle portion comprises a lever, the lever actuable to move the first and second jaws between the closed and open positions.

3. The surgical instrument of claim 2, wherein when the first and second jaws are in the closed position, the spring bias of the spring impedes movement of the lever to prevent movement of the jaws into the open position.

4. The surgical instrument of claim 3, wherein the safety feature further comprises a slider which contacts the spring, wherein movement of the lever pushes the slider against the spring to compress the spring and overcome the spring bias to move the jaws into the open position.

5. The surgical instrument of claim 4, wherein the slider is captured in the recess, wherein movement of the lever translates the slider in the recess.

6. The surgical instrument of claim 2, further comprising a drive rod connected to the lever, the drive rod having a distal end connected to the first and second jaws, wherein the actuation of the lever translates the drive rod to move the first and second jaws between the closed and open positions.

7. The surgical instrument of claim 6, wherein the first jaw comprises a first slot and the second jaw comprises a second slot, wherein the drive rod distal end is connected to the first and second slots and translates along the drive axis with respect to the first and second slots to move the first and second jaws between the open, and closed positions, wherein the lengths of the first and second slots limit the translation of the drive rod.

8. The surgical instrument of claim 7, further comprising an outer shaft extending from the handle portion and pivotably connected to the first and second jaws, wherein the drive rod extends through the outer shaft and translates with respect to the outer shaft.

9. The surgical instrument of claim 8, further comprising a wheel encircling the outer shaft, wherein rotation of the wheel causes rotation of the outer shaft, drive rod, and first and second jaws about the drive axis.

10. A surgical instrument for grasping and cutting of tissue, comprising:
a first jaw having a first grasping portion and a first blade portion;
a second jaw having a second grasping portion and a second blade portion;
a handle portion connected to the first and second jaws, the handle portion actuable to move the first and second jaws between a grasping configuration and an open configuration, wherein when the first and second jaws are in the open configuration, a cutting gap is formed between the first and second blade portions to permit receiving of tissue between the first and second blade portions for cutting of tissue;
wherein the handle portion comprises a safety feature which prevents unintentional movement of the first and second jaws into the open configuration, the safety feature comprising a spring.

11. The surgical instrument of claim 10, wherein the first grasping portion is distal to the first blade portion and the second grasping portion is distal to the second blade portion.

12. The surgical instrument of claim 11, wherein when the first and second jaws are in the grasping configuration, a grasping gap is formed between the first and second grasping portions, and there is no gap between the first and second blade portions.

13. The surgical instrument of claim 11, wherein the first blade portion comprises a first blade surface terminating at a first blade edge and the second blade portion comprises a second blade surface terminating at a second blade edge, wherein the first grasping portion comprises a first grasping surface and the second grasping portion comprises a second grasping surface, wherein the first blade surface is perpendicular to the first grasping surface and the second blade surface is perpendicular to the second grasping surface.

14. The surgical instrument of claim 10, wherein the handle portion comprises a lever, the lever actuable to move the first and second jaws between the grasping and open positions.

15. The surgical instrument of claim 14, further comprising a drive rod connected to the lever, the drive rod having a distal end connected to the first and second jaws, the drive rod defining a drive axis, wherein the actuation of the lever translates the drive rod along the drive axis to move the first and second jaws between the closed, grasping, and open positions.

16. The surgical instrument of claim 15, wherein the first jaw further comprises a first connection portion, the first blade portion positioned between the first grasping portion and the first connection portion, and wherein the second jaw further comprises a second connection portion, the second blade portion positioned between the second grasping portion and the second connection portion, wherein the first and second jaws are connected to one another at a pivot point, the pivot point located on the first and second connection portions.

17. The surgical instrument of claim 16, wherein the first connection portion comprises a first slot and the second connection portion comprises a second slot, wherein the drive rod distal end is connected to the first and second slots and translates along the drive axis with respect to the first and second slots to move the first and second jaws between the open, grasping, and closed positions, wherein the lengths of the first and second slots limit the translation of the drive rod.

18. The surgical instrument of claim 15, further comprising an outer shaft extending from the handle portion and pivotably connected to the first and second jaws, wherein the drive rod extends through the outer shaft and translates with respect to the outer shaft.

19. The surgical instrument of claim 18, further comprising a wheel encircling the outer shaft, wherein rotation of the wheel causes rotation of the outer shaft, drive rod, and first and second jaws about the drive axis.
